(19) United States
(12) Patent Application Publication
(10) Pub. No.: US 2013/0274466 A1
(43) Pub. Date: Oct. 17, 2013

(54) PROCESS FOR THE PREPARATION OF TETRACYCLIC DERIVATIVES AND INTERMEDIATE PRODUCTS USED IN THE PROCESS

(71) Applicants: Boris Gorin, Oakville (CA); Craig Edward Dixon, Brooklin (CA); Yang Qu, Mississauga (CA)

(72) Inventors: Boris Gorin, Oakville (CA); Craig Edward Dixon, Brooklin (CA); Yang Qu, Mississauga (CA)

(73) Assignee: Alphora Research Inc., Mississauga, Ontario (CA)

(21) Appl. No.: 13/876,389

(22) PCT Filed: Sep. 26, 2011
(86) PCT No.: PCT/CA11/50597

§ 371 Date: Jun. 28, 2013

Related U.S. Application Data
(60) Provisional application No. 61/386,815, filed on Sep. 27, 2010.

(51) Int. Cl. C07D 491/044; C07D 207/08; C07D 487/04

(52) U.S. Cl. CPC C07D 491/044; C07D 487/04; C07D 207/08
USPC 540/577; 548/577; 568/745; 548/421

(57) ABSTRACT

A process for preparation of a compound of formula I or a pharmaceutically acceptable salt thereof, is disclosed. The process involves subjecting a compound of formula II to Ullmann-type conditions to effect an intra-molecular ring closure reaction to form the compound of formula I. The different substituents are as described in the specification. Further, the process can provide an alternate route for the synthesis of asenapine from starting materials that can be readily available.

### TECHNICAL FIELD

The specification discloses a process for preparation of tetracyclic derivatives, its pharmaceutically acceptable salts and intermediate products used in the process.

### BACKGROUND

U.S. Pat. No. 4,145,434 discloses that tetracyclic compounds of general formula:

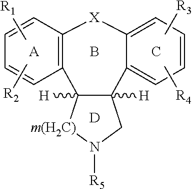

as well as the pharmaceutically acceptable salts and nitrogen oxides thereof, wherein R₁, R₂, R₃ and R₄ each represent hydrogen, hydroxy, halogen, an alkyl (1-6 C) group, an alkoxy or alkylthio group in which the alkyl group contains 1-6 C-atoms, or a trifluoromethyl group, R₅ represents hydrogen, an alkyl group with 1-6 carbon atoms or an aralkyl group with 7-10 carbon atoms, m is the number 1 or 2, X represents oxygen, sulphur, the group NR₆ or the group —CH₂—, and R₆ represents hydrogen or a lower alkyl group (1-4 C), show surprisingly valuable biological activities.

Trans-5-chloro-2-methyl-2,3,3a,12b-tetrahydro-1H-dibenz[2,3:6,7]oxepino[4,5-c]-pyrrole, which is commonly known as asenapine, is a compound having CNS-depressant activity and having antihistamine and antiserotonin activities (U.S. Pat. No. 4,145,434). It has been established that the maleate salt of asenapine, is a broad-spectrum, high potency serotonin, noradrenaline and dopamine antagonist.

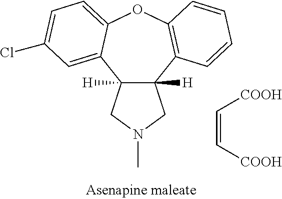

Asenapine exhibits potential antipsychotic activity and may be useful in the treatment of depression (see WO 99/32108). A pharmaceutical preparation suitable for sublingual or buccal administration of asenapine maleate has been described in WO 95/23600.

Alternate route for the synthesis of such tetracyclic compounds, including asenapine, and their pharmaceutically acceptable salts, can be useful.

A general methodology for the preparation of asenapine is disclosed in U.S. Pat. No. 4,145,434. U.S. Pat. No. 7,750,167 (herein the '167 patent) also discloses a process for preparation of asenapine, as well as intermediate products for use in the process. The process disclosed involves an intramolecular ring-closure reaction under Ullmann conditions. Specifically, an E-stilbene derivative is reacted with an azomethine ylide to provide a trans-pyrrolidine derivative having the following formula:

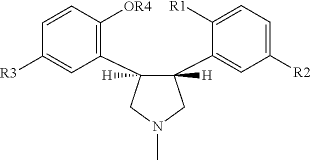

The '167 patent discloses that in the trans-pyrrolidine derivative, shown above, R1 is F, Br or I. Chlorine is not included in the definition of R1. While, R2 and R3 are different and each is selected from H and Cl; and R4 is H or a hydroxyl protecting group. For formation of asenapine, intramolecular ring closure reaction of the trans-pyrrolidine derivative is performed under Ullmann reaction conditions. In the ring closure reaction, R1 (F, Br or I) is substituted by the oxygen atom of the phenol moiety and results in asenapine formation.

The '167 patent discloses that substitution of F, Br or I occurs to form asenapine, but Cl has been excluded from the definition of R1. Moreover, the examples disclosed (examples 3 and 6) in the '167 patent, where Cl atom (as R2) is present on the same aromatic ring as R1 (Br or F), reveal that substitution of R1 (Br or F) occurs, rather than substitution of chlorine.

The Ullmann coupling conditions disclosed in the '167 patent are workable for aryl bromides and aryl iodides, with various ligands, but not reported as viable for aryl chlorides (Altman, R. A. et al., J. Org. Chem., 2008, 73, 284-286; Niu, H. et al., J. Org. Chem., 2008, 73, 7814-7817;Ma, D. et al., Org. Lett., 2003, 5, 3799-3802; Cristau, H.-J. et al., Org. Lett., 2004, 6, 913-916; and references cited therein). This is consistent with the disclosure of the '167 patent, where Cl was excluded from the definition R1, as it would be expected to be unsuitable for the desired aryl ether formation.

There is a need in the art for the preparation of tetracyclic derivatives, including asenapine and its pharmaceutically acceptable salts, via an alternate synthetic route. In addition, there is a need in the art, where asenapine and its pharmaceutically acceptable salts can be prepared from readily available starting materials.

### SUMMARY OF THE INVENTION

In one aspect, the specification discloses a process for preparation of compound of formula I

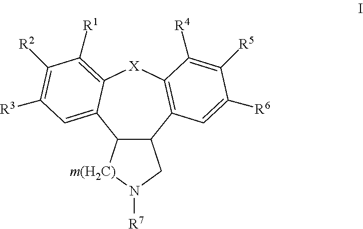

or a pharmaceutically acceptable salt thereof, wherein R¹, R², R³, R⁴, R⁵, R⁶, R⁷, m and X are as described herein; the process comprising subjecting a compound of formula II

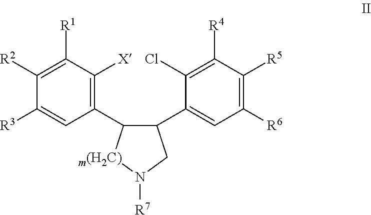

where X′, R¹, R², R³, R⁴, R⁵, R⁶, R⁷, R⁸ and m are as defined herein; to Ullmann-type conditions to effect an intra-molecular ring closure reaction to form the compound of formula I, and optionally converting the compound of formula I to the pharmaceutically acceptable salt thereof.

In another aspect, the specification discloses a compound of formula IV or V, or their respective enantiomer.

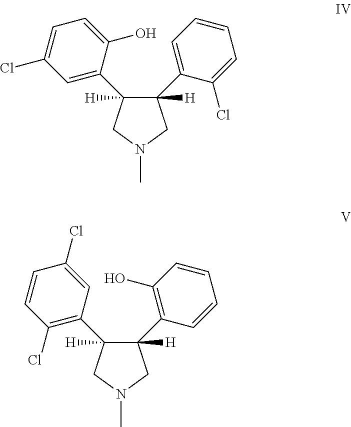 [SMILES: CN1C[C@@H](c2cc(Cl)ccc2O)[C@H](c2ccccc2Cl)C1 ; CN1C[C@@H](c2ccccc2O)[C@H](c2cc(Cl)ccc2Cl)C1]

In a further aspect, the specification discloses a process for the preparation of compound IV, comprising coupling 2-chlorobenzyl bromide with 5-chlorosalicylaldehyde to form trans 2,5′-dichloro-2′-hydroxystilbene; and reacting the trans 2,5′-dichloro-2′-hydroxystilbene with an azomethine ylide to form the compound of formula IV.

### BRIEF DESCRIPTION OF THE DRAWINGS

FIG. 1 shows a ¹H-NMR spectrum of (2-Chloro-benzyl)-phosphonic acid diethyl ester (VII) prepared in accordance with the specification;

FIG. 2 shows a ¹H-NMR spectrum of trans-2,5′-dichloro-2′-hydroxystilbene (VI) prepared in accordance with the specification;

FIG. 3 shows an expansion of a region of the ¹H-NMR spectrum of FIG. 2;

FIG. 4 shows a ¹H-NMR spectrum of trans-N-methyl-2-(2-chlorophenyl)-3-(2-hydroxy-5-chlorophenyl)-pyrrolidine (IV) prepared in accordance with the specification;

FIG. 5 shows a ¹H-NMR spectrum of asenapine hydrobromide prepared in accordance with the specification; and

FIG. 6 shows a mass spectrum of asenapine (III) prepared in accordance with the specification.

### DETAILED DESCRIPTION

As noted above, the '167 patent discloses a process where intramolecular coupling of the phenol moiety occurs with an aryl-halide, where the halide is F, Br or I. Cl is omitted from the list of halides. The literature precedence, noted above, would also lead one to expect that the conditions disclosed in the '167 patent should not be suitable for the intramolecular ring-closure reaction, including the aryl ether formation, when the aryl halide is Cl.

The coupling of aryl chlorides with phenols can be achieved using the so-called Buchwald-Hartwig coupling reaction (Burgos, C. H. et al., Angew. Chem. Int. Ed., 2006, 45, 4321-4326; Sheng, Q. et al., Org. Lett., 2008, 10, 4109-4112). To that end various experiments to effect the desired coupling reaction using Hartwig type conditions were performed by inventor and it was shown that the coupling reaction can be challenging to achieve.

It has now been found that intramolecular coupling reaction of an aryl chloride and phenol moiety, to obtain a ring-closure, can be performed under Ullmann-type reaction conditions to form the tetracyclic derivatives of formula I.

Therefore, in one aspect, the specification discloses a process for preparation of compound of formula I

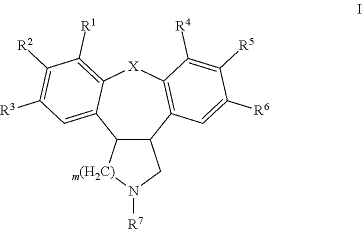

or a pharmaceutically acceptable salt thereof, wherein R¹, R², R³, R⁴, R⁵ and R⁶ each independently is hydrogen, hydroxy, halogen, a C₁₋₆ alkyl, a C₁₋₆ alkoxy, a C₁₋₆ alkylthio or a trifluoromethyl group; R⁷ is H, a C₁₋₆ alkyl or a C₇₋₁₀ aralkyl group; m is 1 or 2; X is O, S or NR⁸, where R⁸ is H or a C₁₋₄ alkyl group; the process comprising subjecting a compound of formula II

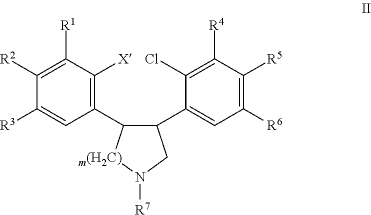

where X′ is OH, SH or —NHR⁸; and R¹, R², R³, R⁴, R⁵, R⁶, R⁷, R⁸ and m are as defined above; under Ullmann-type conditions to effect an intra-molecular ring closure reaction to form the compound of formula I, and optionally converting the compound of formula I to the pharmaceutically acceptable salt thereof.

In one embodiment, the specification discloses a process for the preparation of a compound of formula III

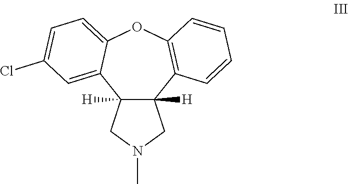 [SMILES: CN1C[C@H]2c3ccccc3Oc3ccc(Cl)cc3[C@@H]2C1]

or its enantiomer, or a pharmaceutically acceptable salt thereof, the process comprising subjecting a compound of formula IV

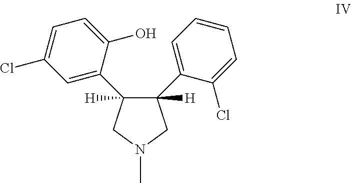 [SMILES: CN1C[C@@H](c2cc(Cl)ccc2O)[C@H](c2ccccc2Cl)C1]

or its enantiomer, under Ullmann-type conditions to effect an intra-molecular ring closure reaction to form the compound of formula III or its enantiomer, and optionally converting the compound of formula III or its enantiomer to the pharmaceutically acceptable salt thereof.

In another embodiment, the specification discloses a process for the preparation of a compound of formula III

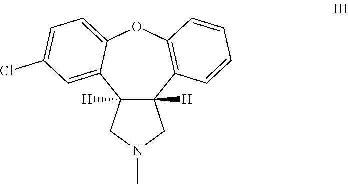 [SMILES: CN1C[C@H]2c3ccccc3Oc3ccc(Cl)cc3[C@@H]2C1]

or its enantiomer, or a racemic mixture thereof, or a pharmaceutically acceptable salt thereof, the process comprising subjecting a compound of formula V

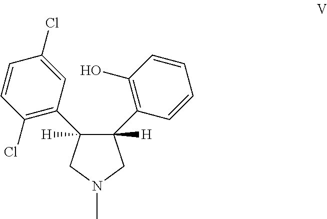 [SMILES: CN1C[C@@H](c2ccccc2O)[C@H](c2cc(Cl)ccc2Cl)C1]

or its enantiomer, under Ullmann-type conditions to effect an intra-molecular ring closure reaction to form the compound of formula III or its enantiomer, and optionally converting the compound of formula III or its enantiomer to the pharmaceutically acceptable salt thereof.

The compounds of formula I may occur in 2 diastereomeric forms, namely as cis-compound or as trans-compound. In the cis-compound, the hydrogen atoms present in the bridge of the compound of formula I, are in the cis-position with respect to each other. In the trans-compound, the two hydrogen atoms are on opposite sides of the bond. The relationship is more clearly seen in the compound of formulas III, IV and V, where a pair of bold and hashed wedged bonds, as shown above, refers to the “trans” diastereoisomer. Each of the compounds may exist as a single enantiomer having the absolute stereochemical configuration indicated by the wedged bonds, or having the opposite absolute configuration, or as a mixture of enantiomers (e.g., racemate) having the relative stereochemical configuration indicated by the wedged bonds.

Both the cis-compounds and the trans-compounds, their enantiomers, racemates, as well as a mixture of both diastereomers, are included amongst the compounds according to the invention. In one embodiment, the compounds of formula I are present in the trans configuration.

Pharmaceutically acceptable salts of the compound of formula I or III would be known to a person of ordinary skill in the art or could be determined based on routine experimentation. Salts of the compound of formula I or III include, for example and without limitation, salts obtained from combination with an organic base, a mineral acid or an organic acid. Examples of such organic bases include, for example and without limitation, trimethylamine, triethylamine and the like. Suitable acid addition salts can be obtained from the treatment with a mineral acid that include, for example and without limitation, hydrochloric acid, hydrobromic acid, phosphoric acid and sulfuric acid, or with an organic acid that include, for example and without limitation, ascorbic acid, citric acid, tartaric acid, lactic acid, maleic acid, malonic acid, fumaric acid, glycolic acid, succinic acid, propionic acid, acetic acid and methane sulfonic acid. In one embodiment, the salt of the compound of formula I or III is a maleate salt.

The C₁₋₆ alkyl group may be, for example, and without limitation, any straight or branched alkyl, for example, methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl or 3-methylpentyl. The C₁₋₄ alkyl group is encompassed by the description of C₁₋₆ alkyl group and is limited to four carbons.

The C₁₋₆ alkoxy group is a C₁₋₆ alkyl group as described above, which is linked to an oxygen atom. For example, and without limitation, the C₁₋₆ alkoxy group may be methoxy, ethoxy, n-propoxy, i-propoxy and the like.

The C₁₋₆ alkylthio group is a C₁₋₆ alkyl group as described above, which is linked to a sulphur atom. For example, and without limitation, the C₁₋₆ alkylthio group may be methylthio, ethylhio, n-propylthio, i-propylthio and the like.

The C₇₋₁₀ aralkyl group may be, for example, and without limitation, a phenylalkyl having 7 to 10 carbon atoms. The phenylalkyl may be, for example, and without limitation, benzyl, phenylethyl, phenylpropyl, 1-methylphenylethyl and the like.

The intramolecular ring closure reaction is performed under Ullmann-type conditions, where, for example, a phenol is coupled to an aryl halide in the presence of a copper compound. In the present case, a compound of formula II, IV or V can be treated with copper(0) powder, with a copper(I) salt or with a copper (II) salt to effect an intramolecular ring-closure reaction. In one embodiment, the Ullmann-type reaction can be carried out in a solvent, in the presence of a base at elevated temperature.

As should be recognized by a skilled worker that different copper compounds can be used for the Ullmann-type coupling reaction. The copper(0) powder, copper(I) salt or copper (II) salt for the Ullmann-type coupling reaction would be known to person of ordinary skill in the art or can be determined. Examples of copper(I) salt or copper (II) salt can include, for example and without limitation, CuI, CuBr, CuCl, Cu(CO)₃ copper(II)carbonate, Cu(OAc)_{Z} copper(II)acetate, Cu(OTf)₂ copper(II)trifluoromethanesulfonate, Cu₂O or CuSO₄. In one embodiment, the Ullmann-type reaction is performed using a copper(I) salt. In another embodiment, the Ullmann-type reaction is performed using CuI or Cu(OAc)₂.

The amount of copper(0) powder, copper(I) salt or copper (II) salt used is not particularly limited and would be known to a person of ordinary skill in the art or can be determined. Examples of the amounts can include 0.1 equivalence (eq.), 0.2 eq., 0.3 eq., 0.4 eq., 0.5 eq., 0.6 eq., 0.7 eq., 0.8 eq., 0.9 eq., 1 eq., 1.1 eq., 1.2 eq., 1.3 eq., 1.4 eq., 1.5 eq., or values in between. In one embodiment, the coupling reaction is carried out with 0.25 eq. or 1 eq.

A ligand may be added for performing the coupling reaction. Examples of ligands used in the Ullmann-coupling reaction would be known to a person of ordinary skill in the art, and can include, without limitation, dimethylethylenediame (DMEDA), triphenylphosphine (TPP), N,N-dimethylglycine (NDMG), tri-t-butylphosphine (tri-tBuP), N-methylglycine, 2,2,4,4-tetramethyl-3,5-heptanedione (TMHD) or 8-hydroxyquinoline. In one embodiment, the ligand is DMEDA or NDMG.

Suitable solvents for use in the Ullmann-type coupling reaction are not particularly limited, and would be known to a person of ordinary skill in the art or can be determined. Examples of a solvent can include, without limitation, dimethylformamide (DMF), dimethylacetamide (DMA), N-methylpyrrolidone (NMP), pyridine, dioxane, toluene, xylene, diethyleneglycoldimethylether (Diglyme), 2-methyltetrahydrofuran, and the like. In one embodiment, the solvent is toluene or dioxane or DMF.

Suitable bases for use in the Ullmann-type coupling reaction are not particularly limited, and would be known to a person of ordinary skill in the art or can be determined. Examples of bases can include, without limitation, K₃PO₄, NaH, K₂CO₃, Cs₂CO₃, pyridine, NaOH, KOH or CsF. In one embodiment, the base is K₃PO₄ or Cs₂CO₃.

Elevated temperature for use in the Ullmann-type coupling reaction is not particularly limited, and would be known to a person of ordinary skill in the art or can be determined. Elevated temperature can include, without limitation, reflux temperature, a temperature range or temperature greater than 100° C. The temperature range can include, for example and without limitation, 90-110° C., 100-120° C., 110-130° C., 120-140° C., 130-150° C., 140-160° C., 150-170° C., 160-180° C., 170-190° C., 180-200° C., 190-210° C. or 200-220° C. Temperature greater than 100° C. can include, without limitation, 110° C., 120° C., 130° C., 140° C., 150° C., 160° C., 170° C., 180° C., 190° C., 200° C., 210° C. or 220° C., or any values in between. In one embodiment, the elevated temperature is 110° C. or 150° C.

The reaction time is not particularly limited and suitable reaction times would be understood and can be determined by those of ordinary skill in the art. In an embodiment of the present invention, the reaction time may be, for example, and without limitation, about 2 hours, about 3 hours, about 4 hours, about 5 hours, about 6 hours, about 8 hours, about 12 hours, about 16 hours, about 20 hours, or about 24 hours, about 48, about 72 hours, about 4 days, about 5 days, about 6 days, about 7 days, about 8 days, about 9 days or about 10 days, or times in between.

In one embodiment, the compound of formula IV is obtained using synthetic Scheme 1. A possible advantage of the synthetic route disclosed in Scheme 1 is that the starting materials can be readily available, and hence, may be economically more feasible.

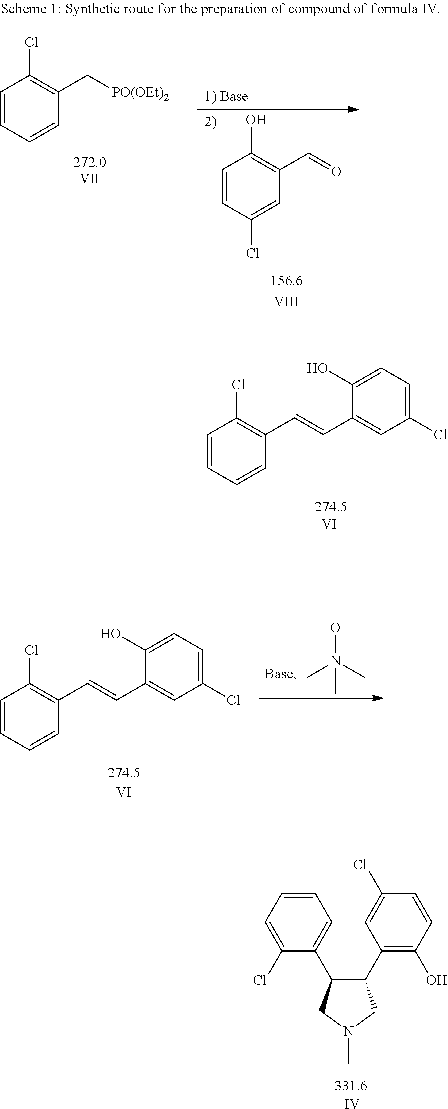

According to Scheme 1, (2-Chloro-benzyl)-phosphonic acid diethyl ester (VII) is used to react with 5-chlorosalicylaldehyde (VIII) in a Wittig type reaction to form trans-2,5′-dichloro-2′-hydroxystilbene (VI). The trans-2,5′-dichloro-2′-hydroxystilbene (VI) is reacted with an azomethine ylide to form trans-N-methyl-2-(2-chlorophenyl)-3-(2-hydroxy-5-chlorophenyl)-pyrrolidine (IV).

Trans-N-methyl-2-(2-chlorophenyl)-3-(2-hydroxy-5-chlorophenyl)-pyrrolidine (IV) is used for the Ullmann-type coupling reaction to form trans-5-chloro-2-methyl-2,3,3a,12b-tetrahydro-1H-dibenz[2,3:6,7]oxepino[4,5-c]-pyrrole (III), as shown in Scheme 2.

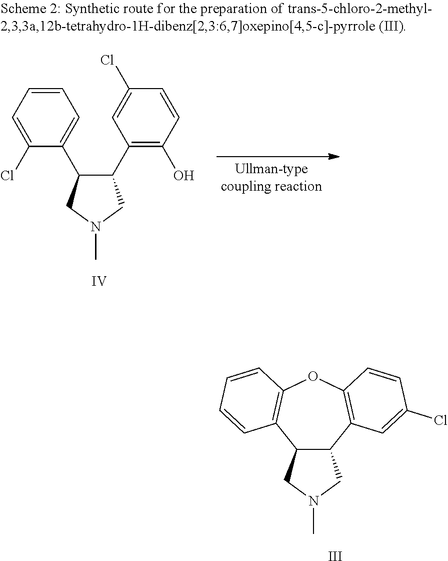

To evaluate the reaction conditions for performing the Ullmann-type coupling reaction, a number of reactions were carried out. The results of the trials are summarized in Table 2 and 3 below.

TABLE 2
Results of the Ullmann-type coupling reaction to produce compound (III)

| RXN | Catalyst | Ligand** | base | solvent | Temp. | Time | | Result |
|---|---|---|---|---|---|---|---|---|
| 9 | CuI (0.25 eq) | DMEDA (0.5 eq) | no base | dioxan | 100 | 24 | H | no pdt observed |
| *10 | CuI (0.25 eq) | DMEDA (0.5 eq) | K₂CO₃ | dioxan | >100 | 72 | H | pdt observed, SM |
| | | | | | | | | remains |
| *11 | CuI (0.25 eq) | DMEDA (0.5 eq) | K₃PO₄ | dioxan + H2O | >100 | 72 | H | pdt observed, SM |
| | | | | | | | | remains |
| 12 | CuI (0.25 eq) | DMEDA (0.5 eq) | no base | toluene | >100 | 24 | H | no pdt observed |
| *13 | CuI (0.25 eq) | DMEDA (0.5 eq) | K₂CO₃ | toluene | >100 | 72 | H | pdt observed, SM |
| | | | | | | | | remains |
| *14 | CuI (0.25 eq) | DMEDA (0.5 eq) | K₃PO₄ | toluene + H2O | >100 | 72 | H | pdt observed, SM |
| | | | | | | | | remains |
| 15 | CuI (0.25 eq) | TCHP (0.5 eq) | K₃PO₄ | toluene | >100 | 24 | H | no pdt observed |
| 16 | CuI (0.25 eq) | TTP (0.5 eq) | K₃PO₄ | toluene | >100 | 24 | H | no pdt observed |
| 17 | CuI (0.25 eq) | TPP (0.5 eq) | K₃PO₄ | toluene | >100 | 24 | H | no pdt observed |
| 18 | CuI (1 eq) | DMEDA (5 eq) | K₃PO₄ | toluene | >100 | 6 | days | see Table 3 |
| 19 | CuI (0.25 eq) | NDMG (0.5 eq) | Cs₂CO₃ | dioxane | >100 | 6 | days | see Table 3 |
| 20 | CuI (0.25 eq) + | DMEDA (0.5 eq) | K₃PO₄ | toluene + H2O | >100 | 24 | H | no pdt observed |
| | (Bu)₄NBr (0.25 eq) | | | | | | | |
| 21 | CuI (0.25 eq) | Tri-tBuP (0.5 eq) | K₃PO₄ | toluene | >100 | 24 | H | no pdt observed |

*Phase transfer catalyst tetrabutyl ammomium bromide (10% mole of CuI) and water were added at 48 hours

TABLE 3
Results of the Ullmann-type coupling reaction over longer time periods.

| RXN | | | | | | RXN | Product peak percentage % | | | | | |
|---|---|---|---|---|---|---|---|---|---|---|---|---|
| # | Catalyst | Ligand | base | solvent | Temp. | Time | 24 hr | 48 hr | 72 hr | 4 days | 5 days | 6 days |
| 18 | CuI (1 eq) | DMEDA (5 eq) | K₃PO₄ | toluene | >110 | 6 days | | | | 27.66 | 31.95 | 44.96 |
| 19 | CuI (0.25 eq) | NDMG (0.5 eq) | Cs₂CO₃ | dioxane | >100 | 6 days | | | | 25.41 | 31.44 | 40.99 |

In an alternate embodiment, the trans-5-chloro-2-methyl-2,3,3a,12b-tetrahydro-1H-dibenz[2,3:6,7]oxepino[4,5-c]-pyrrole (III), can be prepared by performing an Ullmann-type coupling reaction using the E-stilbene derivative (V), as shown in Scheme 3.

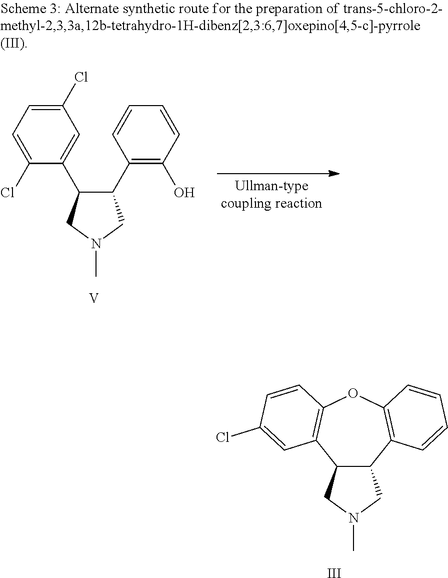

The E-stilbene derivative (X) can be obtained from 2,5-dichlorobenzyl bromide (XI), as shown in Scheme 4. A similar synthetic route can be followed as disclosed in Scheme 1. 2,5-dichlorobenzyl bromide (XI) can be converted into a phosphonate and reacted with salicylaldehyde, followed by reaction with an azomethine ylide to form the E-stilbene derivative (V).

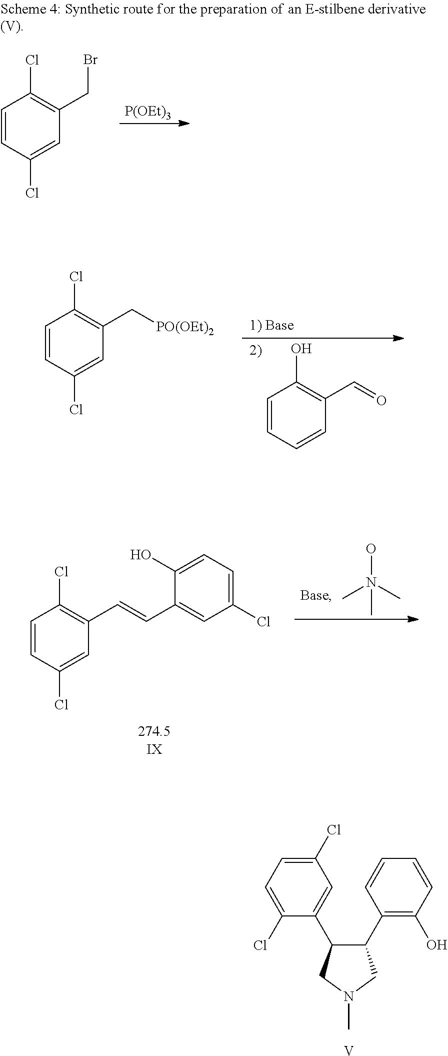

### EXAMPLES

The following examples are illustrative and non-limiting and represent specific embodiments of the present invention.

### Example 1

### (2-Chloro-benzyl)-phosphonic acid diethyl ester (VII)

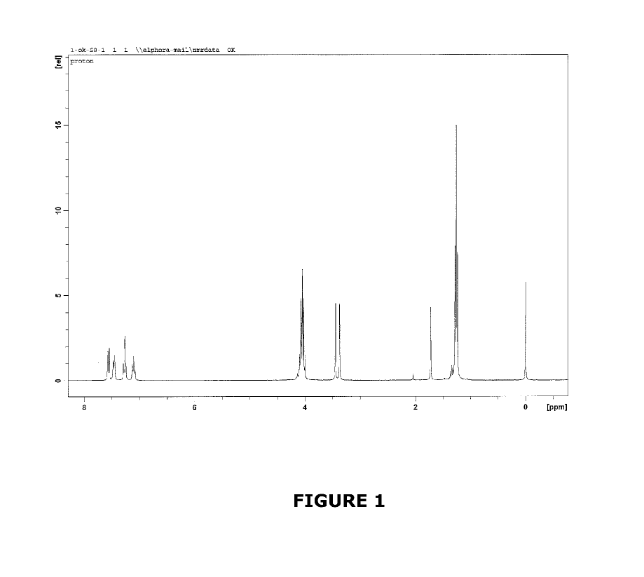

A 0.5 L, single-neck round-bottom flask, equipped with a stirring bar was charged with 12.9 ml (20.55 g; 100 mmol) of 2-chlorobenzyl bromide; 18.4 ml (17.62 g; 106 mmol) of triethyl phosphite and with 75 ml of o-xylene. Stirring was started and the reaction solution was heated to reflux in an oil bath and stirred at that temperature for 20 hours. Upon cooling to room temperature, most of the solvent was evaporated under reduced pressure and the remaining brown oil was purified by filtering through a silica plug (300 g of 40-75 um silica). The plug was conditioned with n-hexanes and eluted with 30% ethyl acetate in n-hexanes (1 L) followed by ethyl acetate (1 L). Appropriate fractions (based on TLC analysis) were pooled and evaporated under reduced pressure to afford colorless oil, 23.91 g (91%). NMR (H¹, CDCl₃) is shown in FIG. 1.

### Example 2

### Trans-2,5′-dichloro-2′-hydroxystilbene (VI)

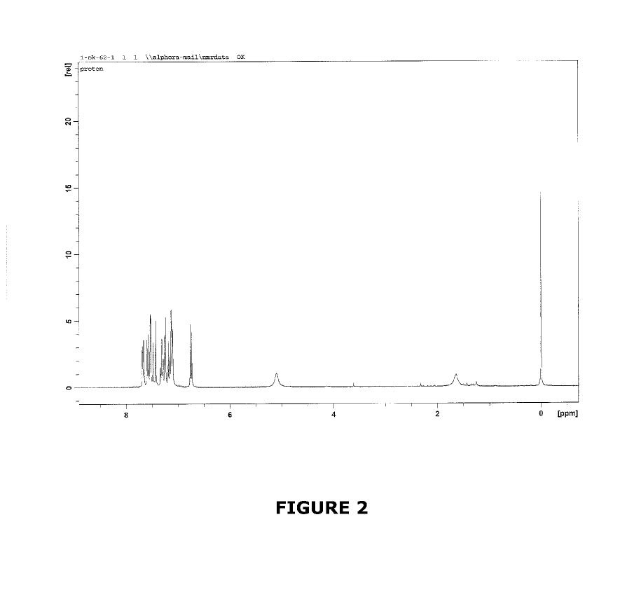

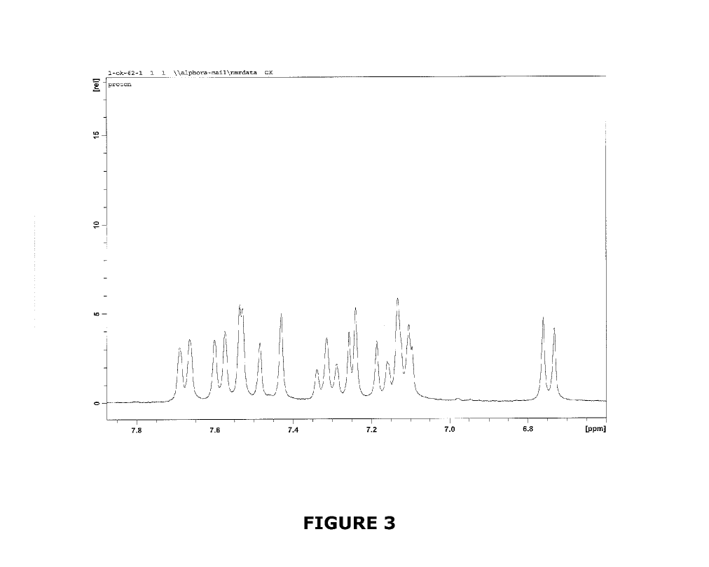

A 1 L, three-neck round-bottom flask equipped with a stirring bar, nitrogen inlet, dropping funnel and thermometer was charged with 23.64 g (90 mmol) of (2-chloro-benzyl)-phosphonic acid diethyl ester; 14.09 g (90 mmol) of 5-chlorosalicylaldehyde and with 200 ml of anhydrous THF. Dropping funnel was charged with 200 ml of 1M solution of potassium tert-butoxide in THF (200 mmol of tert-butoxide). Stirring was started and the reaction solution was cooled down to 10° C. in an ice-water bath. Tert-butoxide solution was added drop-wise over 1 hour while maintaining the temperature of the reaction medium below 35° C. At that time the bath was removed and red reaction solution was allowed to reach room temperature for 24 hours. 200 ml of water were added in one portion followed by 500 ml of ethyl acetate. Organic phase was separated and washed with 250 ml of brine. The resultant solution was dried over sodium sulfate and evaporated under reduced pressure to furnish brown oil which crystallized spontaneously to afford 17.42 g of tan solid (73%). NMR (H¹, CDCl₃) are shown in FIGS. 2 and 3.

### Example 3

### Trans-N-methyl-2-(2-chlorophenyl)-3-(2-hydroxy-5-chlorophenyl)-pyrrolidine (IV)

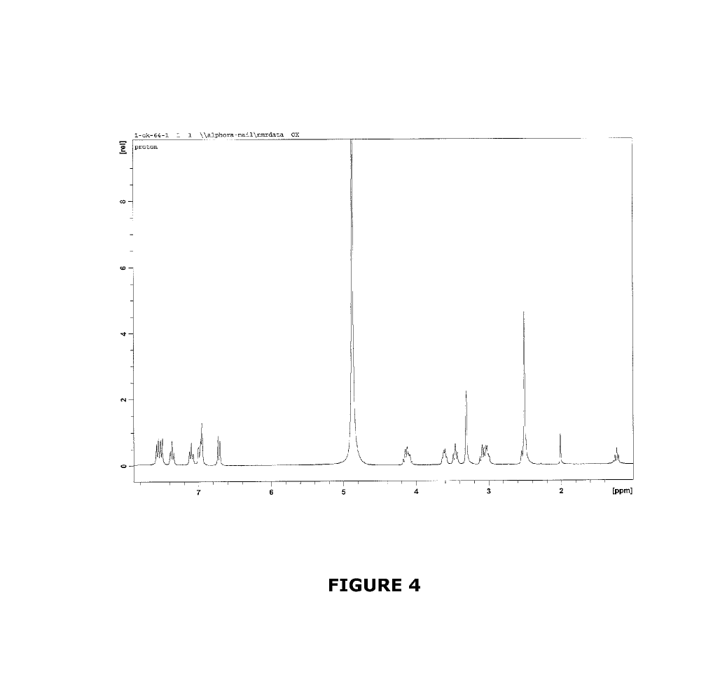

A 1 L, three-neck round-bottom flask equipped with a stirring bar, nitrogen inlet, dropping funnel and thermometer was charged with 6.89 g (26 mmol) of trans-2,5′-dichloro-2′-hydroxystilbene; 4.33 g (39 mmol) of trimethylamine N-oxide dihydrate and with 100 ml of anhydrous THF. Dropping funnel was charged with 240 ml of 1M solution of lithium bis(trimethylsilyl)amide solution in THF (240 mmol of the amide). Stirring was started and lithium bis(trimethylsilyl)amide solution was added drop-wise to the reaction mixture over 40 min. By the end of addition, temperature of the reaction medium rose to 32° C. Funnel was changed to a reflux condenser and reaction mixture was heated to reflux in an oil bath and stirred at that temperature for 18 hours. At that point, it was cooled down to room temperature and 200 ml of water were added in one portion followed by 600 ml of ethyl acetate. Aqueous layer was separated and its pH was adjusted to 8 by drop-wise addition of 18% (w/w) hydrochloric acid. The resultant emulsion was back-extracted with 200 ml of ethyl acetate. Combined organic phases were washed with 200 ml of brine, dried (sodium sulfate) and evaporated under reduced pressure to furnish brown oil which was purified by a silica plug (180 g of 40-75 um silica). The plug was conditioned with n-hexanes and eluted with 30% ethyl acetate in n-hexanes (0.5 L) followed by ethyl acetate (0.5 L). Appropriate fractions of eluate (based on TLC analysis) were pooled and evaporated under reduced pressure to afford yellowish viscous oil, 6.37 g (76%). NMR (H¹, CDCl₃) is shown in FIG. 4.

### Example 4

### Asenapine Hydrobromide

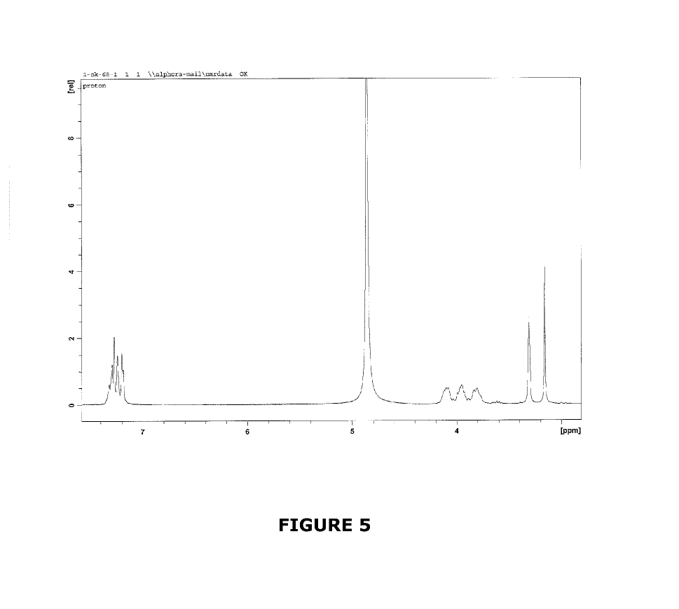

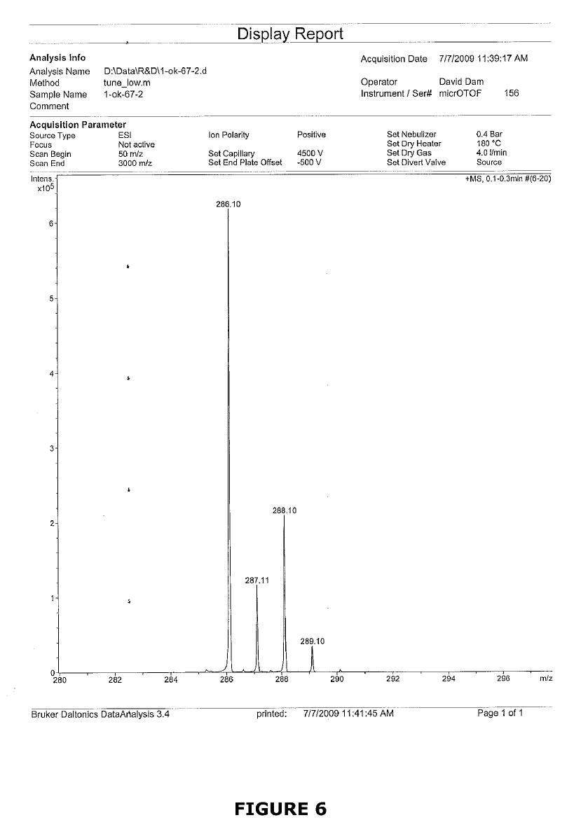

A 0.5 L, three-neck round-bottom flask equipped with a stirring bar, nitrogen inlet, and reflux condenser was charged with 6.00 g (18.6 mmol) of trans-N-methyl-2-(2-chlorophenyl)-3-(2-hydroxy-5-chlorophenyl)-pyrrolidine and with 50 ml of 1,4-dioxane. Stirring was started and 7.28 g (22.3 mmol) of cesium carbonate, 0.90 g (4.7 mmol) of cuprous iodide, 0.50 g (4.7 mmol) of N,N-dimethylglycine were added sequentially to the reaction solution. The resultant brown mixture was heated to reflux and stirred at that temperature for 90 hours. Upon cooling, greenish reaction mixture was filtered through a Celite pad ( ) which was washed subsequently with 1,4-dioxane (2×50 ml). Combined filtrates were evaporated under reduced pressure to afford brown oil. This oil was charged to a 250 ml single-neck round-bottom flask equipped with a stirring bar. 70 ml of ethanol were added and oil was dissolved in the solvent. Stirring was started and 2.2 ml of 48% hydrobromic acid were added to the solution in one portion. After a couple of minutes of stirring at room temperature, creamy solid began to precipitate. The resultant suspension was stirred at that temperature for 8 hours and at that point solid phase was separated on a glass filter funnel, washed with 50 ml of isopropanol (IPA) followed by 50 ml of methyl tert-butyl ether (MTBE). The solid was dried out in a vacuum oven at ambient temperature for 2 hours; off-white crystals, 4.36 g (64%). NMR (H¹, methanol-d₄) is shown in FIG. 5; MS (infusion of 1 ug/ml solution of the compound in MeOH into a Bruker microTOF instrument operated in positive ESI mode) is shown in FIG. 6.

### Example 5

### Asenapine Maleate

To obtain asenapine maleate, the bromide salt formed in example 4 (13.4 grams, 0.036 mol) can be neutralized with 28% aqueous solution of ammonia in water (70 ml). The free base is extracted with ethylacetate (2*50 ml) and the organic layer washed with saturated NaCl, concentrated under reduced pressure to provide 10.4 grams of asenapine, as the free base. The free base can be dissolved in ethanol (20.8 ml) and heated to 60° C. Maleic acid (4.65 grams 0.040 mol) is added and the mixture is stirred for 2 h at −15° C., whereupon the maleate can precipitate. The crystals can be collected by filtration, washed with ethanol (20.8 ml) and diisopropylether (20.8 ml). To obtain the desired polymorph, the isolated crystals can be dissolved in ethanol (18 ml) and water (2 ml) at 55° C. The temperature reduced to 20° C. and the desired polymorph can precipitate slowly over 48 h. The crystals can be filtered, washed with ethanol (10 ml) and dried under reduced pressure at 40° C.

### Example 6

### Trans-N-methyl-2-(2-chlorophenyl)-3-(2-hydroxy-5-chlorophenyl)-pyrrolidine (IV)

5-chlorosalicylaldehyde (5.0 g, 31.9 mmol, 1.0 equiv) was transferred to a 150 ml round bottom flask and was dissolved in toluene (75 ml, 15 parts). (2-chloro-benzyl)-phosphonic acid diethyl ester (10.1 g, 38.3 mmol, 1.2 equiv) was added and the resulting mixture stirred at room temperature for 15 min to get a yellow solution. Potassium tert-butoxide (1 M solution) in tetrahydrofuran (71.8 ml, 71.8 mmol, 2.25 equiv) was transferred to a 500 ml 3 N round bottom flask under nitrogen, and was cooled to 15-20° C. in a cold water bath. The 5-chlorosalicylaldehyde/(2-chloro-benzyl)-phosphonic acid diethyl ester solution was added slowly over 1 hr keeping the internal temperature below 20° C. The resulting amber solution stirred to room temperature over 1 hr and was complete by TLC. Hydrochloric acid 2 M (25 ml, 5 parts) was added slowly via addition funnel and stirred at room temperature for 10 min. The layers were separated, and the organic layer was washed with saturated sodium bicarbonate solution (25 ml, 5 parts). The organic layer was concentrated to 5 parts (25 ml), and toluene (75 ml, 15 parts) was added. The solution was concentrated to 5 parts and solids started forming. Toluene (75 ml, 15 parts) was added and the mixture was concentrated to 5 parts (solids observed). Toluene (25 ml, 5 parts) was added and the yellow solution containing trans-2,5′-dichloro-2′-hydroxystilbene (VI) was stored at room temperature for 18 hrs.

The above trans-2,5′-dichloro-2′-hydroxystilbene (VI) solution in toluene was filtered into a 250 ml round bottom flask containing trimethylamine oxide (3.12 g, 41.5 mmol, 1.3 equiv). The bright orange mixture was stirred at room temperature for 15 min and was then concentrated to 5 parts (25 ml). Lithium bis(trimethylsilyl)amide (LiHMDS, 1M) in toluene (159.6 ml, 159.6 mmol, 5 equiv) was transferred to a 500 ml round bottom flask under nitrogen and was heated to 80° C. The solution of trans-2,5′-dichloro-2′-hydroxystilbene (VI) and trimethylamine oxide was slowly added to the LiHMDS/toluene solution over 30 min, while maintaining the temperature between 80-90° C. The resulting yellow/brown solution stirred at ˜85° C. for 1 hr and was complete by TLC. The mixture was cooled to room temperature and water (67.5 ml, 13.5 parts) was added. The mixture agitated at room temperature for 18 hr and the layers were separated. The organic layer was washed with water (2×67.5 ml) and was concentrated to 5 parts (25 ml). 2-propanol (50 ml, 10 parts) was added and the mixture was concentrated to 5 parts. 2-propanol (50 ml, 10 parts) was added and the mixture was concentrated to 5 parts (solids observed). 2-propanol (50 ml, 10 parts), then water (50 ml, 10 parts) was added. The resulting suspension was heated to reflux to get a clear yellow solution. The mixture was slowly cooled to room temperature in the oil bath for 18 hr (solids observed at 60° C.). The suspension was filtered and the solids were washed with 75% water in 2-propanol (2×15 ml). The solids dried on the filter under vacuum (nitrogen) for 1 hr to get 8.31 g of trans-N-methyl-2-(2-chlorophenyl)-3-(2-hydroxy-5-chlorophenyl)-pyrrolidine (IV) as a white to off-white solid.

### Example 7

### Asenapine Maleate

To a 1 L three-neck round-bottom flask was added cesium carbonate (27.9 g, 85.7 mmol, 1.2 equiv), copper(I) acetate (2.2 g, 71.8 mmol, 0.25 equiv), N,N-dimethylglycine (7.4 g, 71.4 mmol, 1 equiv) and DMF (160 mL, 7 parts). The suspension was placed under nitrogen and was heated to 140° C. A solution of trans-N-methyl-2-(2-chlorophenyl)-3-(2-hydroxy-5-chlorophenyl)-pyrrolidine (IV) (23.0 g, 71.4 mmol, 1 equiv) in dimethyl formamide (DMF)_(70 mL, 3 parts) was added over 20 min (temperature ranged from 137-147° C.). The resulting suspension was stirred for 3.5 h at 140° C. Once complete by TLC, the reaction mixture was cooled to room temperature and filtered. Methyl tert-butyl ether (MTBE) (115 mL, 5 parts) was used to wash the filter cake. Deionized water (115 mL, 5 parts) was then added to the combined filtrate and washed (exotherm to 35° C.) and the mixture was stirred for 10 min. The layers were separated and the aqueous layer was extracted two additional times with MTBE (2×115 mL, 2×5 parts). The combined organic layer was concentrated to a final volume of 23 mL (1 part) and dichloromethane (11.5 mL, 0.5 parts) was added. The resulting solution was charged onto a 340 g Biotage® SNAP Cartridge (KP Sil, containing 340 g silica gel, 15:1 silica gel:5) pre-conditioned with 3 column volumes (CV) dichloromethane (1.35 L; one column volume, CV, equals 450 mL). Dichloromethane (11.5 mL, 0.5 parts) was used as rinse. Asenapine (III) was eluted with 3 CV dichloromethane (1.35 L) followed by 10 CV of a premixed solution of 40% (v/v) heptane and 58% (v/v) ethyl acetate and 2% (v/v) triethylamine. Solvent was eluted at a rate of 80 ml/min. The first 1.35 L of eluent was collected to waste followed by 10 fractions of 450 ml each. All fractions containing asenapine (III) by TLC were combined and concentrated to dryness under vacuum on a rotary evaporator at 40° C. Asenapine (III) 13.6 g was isolated as brown oil (62% yield, corrected for purity only). Asenapine (III) was co-evaporated with anhydrous ethanol (3×276 mL, 3×12 parts with respect to 5) and then dissolved in ethanol (55 mL, 4 parts with respect to 6) once again. A solution of maleic acid (6.1 g, 52.2 mmol, 1.1 equiv with respect to 6) in anhydrous ethanol (33 mL, 2.4 parts with respect to 6) was added at room temperature. The resulting solution was stirred at room temperature for 16 h and then at approximately 0° C. for 3 h. The suspension was filtered and the solids were washed with ethanol (13 mL, 1 part with respect to 6) that had been pre-cooled to 0° C. The solids were dried on the filter under vacuum and under a stream of nitrogen for 2 h to afford 13.3 g of Asenapine maleate as an off-white solid (46% yield).

Following the methodology disclosed, a number of different compounds according to the invention can be prepared. Table 4 provides a list of different substituents that can be present in the compound of formula I and which can be prepared according to the invention.

TABLE 4
Substituents on compound of formula I

| Sub- | | | | | | | | | |
|---|---|---|---|---|---|---|---|---|---|
| stit- | | | | | | | | | |
| uent | R¹ | R² | R³ | R⁴ | R⁵ | R⁶ | R⁷ | R⁸ | X |
| A | CH₃ | H | H | CH₃ | H | H | CH₃ | — | O |
| B | H | OCH₃ | H | H | H | H | CH₃ | — | O |
| C | H | H | Cl | H | H | H | CH₃ | CH₃ | N |
| D | CH₃ | H | Cl | H | OCH₃ | H | CH₃ | CH₃ | N |
| E | H | H | Cl | H | H | Propyl | Ethyl | CH₃ | N |

1. A process for preparation of a compound of formula I 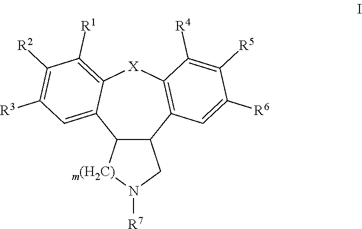
or a pharmaceutically acceptable salt thereof, wherein
R¹, R², R³, R⁴, R⁵ and R⁶ each independently is hydrogen, hydroxy, halogen, a C₁₋₆ alkyl, a C₁₋₆ alkoxy, a C₁₋₆ alkylthio or a trifluoromethyl group;
R⁷ is H, a C₁₋₆ alkyl or a C₇₋₁₀ aralkyl group;
m is 1 or 2;
X is O, S or NR⁸, where R⁸ is H or a C₁₋₄ alkyl group;
the process comprising:
subjecting a compound of formula II 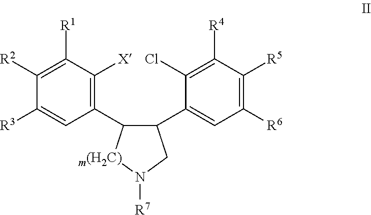
where
X′ is OH, SH or —NHR⁸; and
R¹, R², R³, R⁴, R⁵, R⁶, R⁷, R⁸ and m are as defined above;
to Ullmann-type conditions to effect an intra-molecular ring closure reaction to form the compound of formula I, and
optionally converting the compound of formula I to the pharmaceutically acceptable salt thereof.

2. A process for the preparation of a compound of formula III 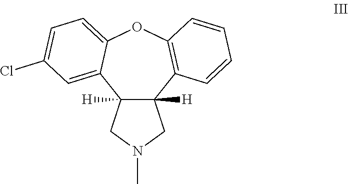 [SMILES: CN1C[C@H]2c3ccccc3Oc3ccc(Cl)cc3[C@@H]2C1]
or its enantiomer, or a pharmaceutically acceptable salt thereof, the process comprising:
subjecting a compound of formula IV 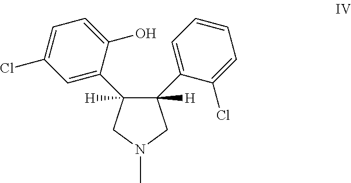 [SMILES: CN1C[C@@H](c2cc(Cl)ccc2O)[C@H](c2ccccc2Cl)C1]
or its enantiomer, to Ullmann-type conditions to effect an intra-molecular ring closure reaction to form the compound of formula III or its enantiomer, and
optionally converting the compound of formula III or its enantiomer to the pharmaceutically acceptable salt thereof.

3. The process according to claim 2, wherein the compound of formula IV is obtained by:
coupling (2-chloro-benzyl)-phosphonic acid diethyl ester (VII) with 5-chlorosalicylaldehyde (VIII) to form trans-2,5′-dichloro-2′-hydroxystilbene (VI); and
reacting the trans-2,5′-dichloro-2′-hydroxystilbene (VI) with an azomethine ylide to form the compound of formula IV.

4. (canceled)

5. The process according to claim 1, wherein the Ullmann-type condition to effect the intramolecular ring-closure reaction is carried out using CuI or copper (I) acetate.

6. The process according to claim 1, wherein the intramolecular ring-closure is carried out at elevated temperature.

7. The process according to claim 6, wherein the intramolecular ring-closure reaction is carried out by refluxing.

8. The process according to claim 6, wherein the intramolecular ring-closure reaction is carried out at a temperature greater than 100° C.

9. The process according to claim 1, wherein the Ullmann-type coupling reaction is carried in a solvent, and wherein the solvent is toluene or dioxane or DMF.

10. The process according to claim 1, wherein the Ullmann-type coupling reaction is carried in the presence of a ligand, and wherein the ligand is DMEDA or NDMG.

11. The process according to claim 1, wherein the Ullmann-type coupling reaction is carried in the presence of a base, and wherein the base is K₃PO₄ or Cs₂CO₃.

12. The process according to claim 1, wherein the Ullmann-type coupling reaction is carried out for about 3, 4, 5, or 6 hours.

13. The process according to claim 1, wherein the pharmaceutically acceptable salt is prepared in the form of its maleate salt.

14. The compound

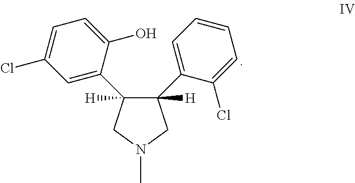 [SMILES: CN1C[C@@H](c2cc(Cl)ccc2O)[C@H](c2ccccc2Cl)C1]

15. (canceled)

16. A process for preparing the compound of formula IV, comprising: 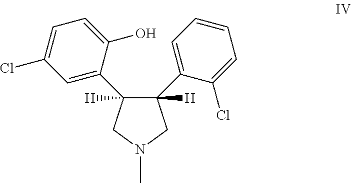 [SMILES: CN1C[C@@H](c2cc(Cl)ccc2O)[C@H](c2ccccc2Cl)C1]
coupling (2-chloro-benzyl)-phosphonic acid diethyl ester (VII) with 5-chlorosalicylaldehyde to form trans 2,5′-dichloro-2′-hydroxystilbene; and
reacting the trans 2,5′-dichloro-2′-hydroxystilbene with an azomethine ylide to form the compound of formula IV.

17. The process according to claim 16, wherein 2-chlorobenzyl bromide is converted into (2-chloro-benzyl)-phosphonic acid diethyl ester (VII) for coupling with 5-chlorosalicylaldehyde, to form trans 2,5′-dichloro-2′-hydroxystilbene.

18. The compound

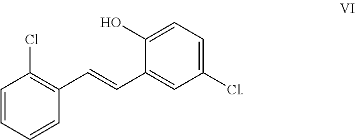 [SMILES: Oc1ccc(Cl)cc1/C=C/c1ccccc1Cl]

19. (canceled)
